(19) United States
(12) Patent Application Publication
(10) Pub. No.: US 2011/0077686 A1
(43) Pub. Date: Mar. 31, 2011

(54) INTERSPINOUS PROCESS IMPLANT HAVING A COMPLIANT SPACER

(75) Inventors: Tanmay Mishra, Mountain View, CA (US); Lauren I. Lyons, San Francisco, CA (US); Christopher U. Phan, San Leandro, CA (US)

(73) Assignee: KYPHON SARL, Neuchatel (CH)

(21) Appl. No.: 12/569,513

(22) Filed: Sep. 29, 2009

(51) Int. Cl. A61B 17/70

(52) U.S. Cl. 606/249

(57) ABSTRACT

Medical devices for the treatment of spinal conditions are described herein. The medical device of this invention includes a spacer that is disposed between adjacent spinous processes and has a layer of a soft or compliant material. The layer is preferably thicker along those portions of the spacer directly contacting the adjacent spinous processes and is preferably thinner or non-existent adjacent to the anterior portion of the support member. This preferred asymmetry of the compliant layer allows the spacer to be seated between spinous processes as anteriorly as possible.

### BACKGROUND

This invention relates generally to the treatment of spinal conditions, and more particularly, to the treatment of spinal stenosis using devices for implantation between adjacent spinous processes.

The clinical syndrome of neurogenic intermittent claudication due to lumbar spinal stenosis is a frequent source of pain in the lower back and extremities, leading to impaired walking, and causing other forms of disability in the elderly. Although the incidence and prevalence of symptomatic lumbar spinal stenosis have not been established, this condition is the most frequent indication of spinal surgery in patients older than 65 years of age.

Lumbar spinal stenosis is a condition of the spine characterized by a narrowing of the lumbar spinal canal. With spinal stenosis, the spinal canal narrows and pinches the spinal cord and nerves, causing pain in the back and legs. It is estimated that approximately 5 in 10,000 people develop lumbar spinal stenosis each year. For patients who seek the aid of a physician for back pain, approximately 12%-15% are diagnosed as having lumbar spinal stenosis.

Common treatments for lumbar spinal stenosis include physical therapy (including changes in posture), medication, and occasionally surgery. Changes in posture and physical therapy may be effective in flexing the spine to decompress and enlarge the space available to the spinal cord and nerves—thus relieving pressure on pinched nerves. Medications such as NSAIDS and other anti-inflammatory medications are often used to alleviate pain, although they are not typically effective at addressing spinal compression, which is the cause of the pain.

Surgical treatments are more aggressive than medication or physical therapy, and in appropriate cases surgery may be the best way to achieve lessening of the symptoms of lumbar spinal stenosis. The principal goal of surgery is to decompress the central spinal canal and the neural foramina, creating more space and eliminating pressure on the spinal nerve roots. The most common surgery for treatment of lumbar spinal stenosis is direct decompression via a laminectomy and partial facetectomy. In this procedure, the patient is given a general anesthesia as an incision is made in the patient to access the spine. The lamina of one or more vertebrae is removed to create more space for the nerves. The intervertebral disc may also be removed, and the adjacent vertebrae may be fused to strengthen the unstable segments. The success rate of decompressive laminectomy has been reported to be in excess of 65%. A significant reduction of the symptoms of lumbar spinal stenosis is also achieved in many of these cases.

Alternatively, the vertebrae can be distracted and an interspinous process device implanted between adjacent spinous processes of the vertebrae to maintain the desired separation between the vertebral segments. Such interspinous process implants typically work for their intended purposes, but some could be improved. Where the spacer portion of the implant is formed from a hard material, point loading of the spinous process can occur due to the high concentration of stresses at the point where the hard material of the spacer contacts the spinous process. This may result in excessive subsidence of the spacer into the spinous process. In addition, if the spinous process is osteoporotic, there is a risk that the spinous process could fracture when the spine is in extension.

Thus, a need exists for improvements in certain current interspinous process devices.

### SUMMARY OF THE INVENTION

The interspinous process implant of this invention includes a spacer that is disposed between adjacent spinous processes and has a layer of a soft or compliant material. Such a layer minimizes the high stress concentration between the spacer and the spinous process and thus improves the point loading characteristics of the spacer on the spinous process. This minimizes subsidence and also reduces the risk of fracture. The durometer of the layer is chosen to provide a sufficient cushion for the spinous process without minimizing the distraction capability of the spacer. Preferably, the compliant layer is located around the spacer such that the layer is thicker along those portions of the spacer directly contacting the adjacent spinous processes and is thinner adjacent to the anterior portion of the spacer. This asymmetry of the compliant layer allows the spacer to be seated between spinous processes as anteriorly as possible. Alternatively, the compliant layer may be located symmetrically (i) about the entire spacer, or (ii) such that the layer is located only along those portions of the spacer adapted to be directly in contact with the spinous processes, or (iii) such that the compliant layer is thicker along the superior and inferior portions of the spacer but such that there is also a thin layer around the anterior and posterior portions of the spacer, or (iv) about entire implant.

In an alternative embodiment, a layer of soft or compliant material can be located within the spacer of the interspinous process implant as a separate core, which may have various cross sections, such as a circle or rectangle. As with the compliant layer described above, the durometer of the material can be adjusted in such a way so as to minimize the point loading on the spinous process and allow the core to take up some of the load. Again, this would minimize subsidence and reduce the risk of fracturing the spinous process.

### BRIEF DESCRIPTION OF THE DRAWINGS

FIG. 1 is a side perspective view of one embodiment of an interspinous process implant shown in a collapsed configuration which may include the spacer of this invention;

FIG. 2 is a cross-sectional perspective view of the implant of FIG. 1 taken along line 2-2;

FIG. 3 is a side perspective view of the implant of FIGS. 1 and 2 shown in a deployed configuration;

FIG. 4 is cross-sectional perspective view of the implant of FIG. 3 taken along line 4-4;

FIG. 5 is a cross-sectional view of the implant of FIG. 1 similar to the view shown in FIG. 2 but with a compliant layer disposed around the spacer;

FIG. 6 is a schematic cross-sectional view of one embodiment of the spacer of this invention disposed between adjacent spinous processes;

FIG. 7 is a schematic cross-sectional view, similar to the view of FIG. 6, of yet another embodiment of the spacer of this invention;

FIG. 8 is a schematic cross-sectional view, similar to the view of FIG. 6, of still another embodiment of the spacer of this invention;

FIG. 9 is a schematic cross-sectional view of an implant, similar to the view of FIG. 6, of a further embodiment of the spacer of this invention;

FIG. 10 is a cross-sectional perspective view, similar to the view shown in FIG. 5, of another embodiment of the spacer of this invention;

FIG. 11 is another cross-sectional view of the embodiment of the spacer of this invention shown in FIG. 10 taken along line 11-11;

FIG. 12 is a cross-sectional view, similar to the view of FIG. 11, of yet another embodiment of the spacer of this invention;

FIG. 13 is a perspective view of still another interspinous process implant that may incorporate the spacer of this invention; and

FIG. 14 is a perspective view of yet another interspinous process implant that may incorporate the spacer of this invention.

### DETAILED DESCRIPTION

As used in this specification and the appended claims, the singular forms “a,” “an” and “the” include plural referents unless the context clearly dictates otherwise. Thus, for example, the term “a member” is intended to mean a single member or a combination of members, “a material” is intended to mean one or more materials, or a combination thereof. Furthermore, the words “proximal” and “distal” refer to directions closer to and away from, respectively, an operator (e.g., surgeon, physician, nurse, technician, etc.) who would insert the medical device into the patient, with the tip-end (i.e., distal end) of the device inserted inside a patient's body first. Thus, for example, the implant end first inserted inside the patient's body would be the distal end of the implant, while the implant end to last enter the patient's body would be the proximal end of the implant.

As used in this specification and the appended claims, the term “body” means a mammalian body. For example, a body can be a patient's body, or a cadaver, or a portion of a patient's body or a portion of a cadaver.

As used in this specification and the appended claims, the term “parallel” describes a relationship, given normal manufacturing or measurement or similar tolerances, between two geometric constructions (e.g., two lines, two planes, a line and a plane, two curved surfaces, a line and a curved surface or the like) in which the two geometric constructions are substantially non-intersecting as they extend substantially to infinity. For example, as used herein, a line is said to be parallel to a curved surface when the line and the curved surface do not intersect as they extend to infinity. Similarly, when a planar surface (i.e., a two-dimensional surface) is said to be parallel to a line, every point along the line is spaced apart from the nearest portion of the surface by a substantially equal distance. Two geometric constructions are described herein as being “parallel” or “substantially parallel” to each other when they are nominally parallel to each other, such as for example, when they are parallel to each other within a tolerance. Such tolerances can include, for example, manufacturing tolerances, measurement tolerances or the like.

As used in this specification and the appended claims, the term “normal” describes a relationship between two geometric constructions (e.g., two lines, two planes, a line and a plane, two curved surfaces, a line and a curved surface or the like) in which the two geometric constructions intersect at an angle of approximately 90 degrees within at least one plane. For example, as used herein, a line is said to be normal to a curved surface when the line and the curved surface intersect at an angle of approximately 90 degrees within a plane. Two geometric constructions are described herein as being “normal” or “substantially normal” to each other when they are nominally normal to each other, such as for example, when they are normal to each other within a tolerance. Such tolerances can include, for example, manufacturing tolerances, measurement tolerances or the like.

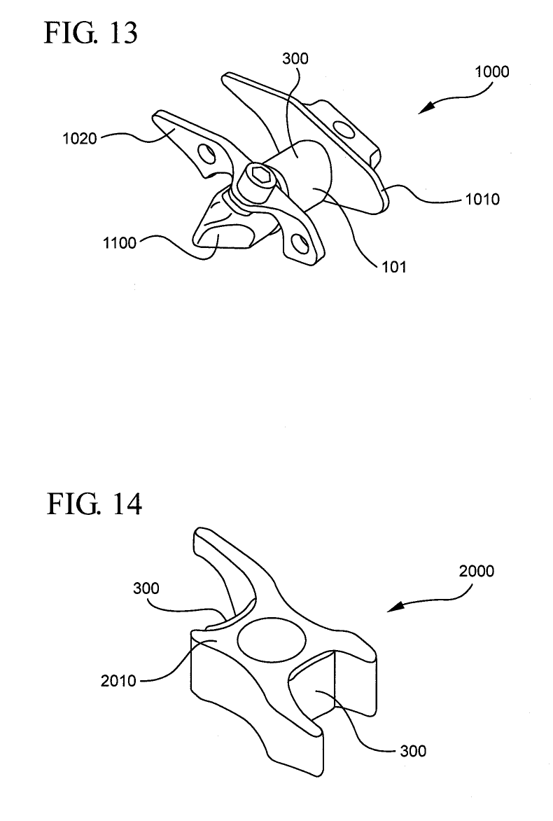

In one embodiment of the interspinous process implant of the invention, the implant includes a spacer that defines a longitudinal axis and is configured to be implanted at least partially into a space between adjacent spinous processes. The implant also has a first retention member and a second retention member. An axial force is exerted along the longitudinal axis such that each of the first retention member and the second retention member plastically expand in a direction transverse to the longitudinal axis. When plastically expanded, each of the first retention member and the second retention member has a greater outer perimeter than an outer perimeter of the support member. The implant configuration is shown in more detail in U.S. Patent Application Publication No. 2007/0225807, the entire contents of which are hereby expressly incorporated herein by reference. Although the interspinous process implant spacer of this invention is described specifically in connection with the configuration shown in U.S. Patent Application Publication No. 2007/0225807, it is to be understood that the invention described herein can be used in connection with other configurations for an interspinous process implant. For example, the invention described herein can be used in connection with the various interspinous process implants having a relatively hard spacer shown in U.S. Patent Application Publication Nos. 2008/0039859 and 2008/0086212, the entire contents of which are hereby expressly incorporated herein by reference. See also FIGS. 13 and 14.

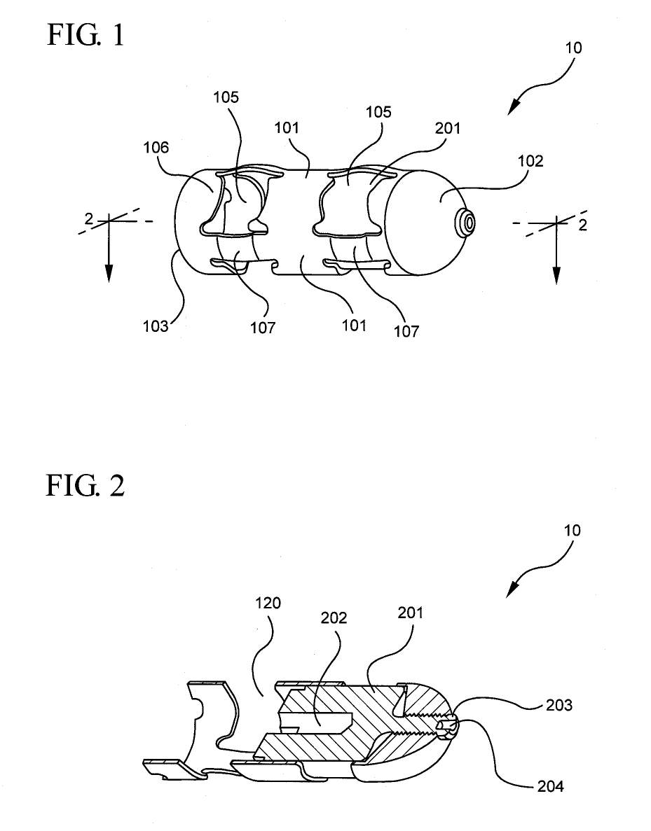

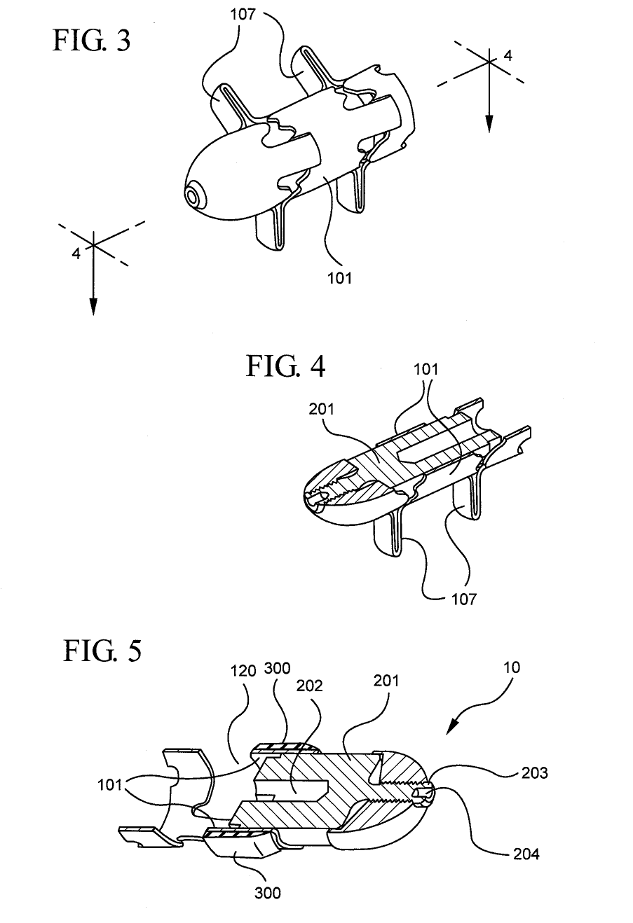

FIGS. 1-4 illustrate an interspinous process implant 10 that may incorporate the spacer of this invention. Implant 10 can be moved between a collapsed configuration, as shown in FIGS. 1 and 2, and a deployed configuration, as shown in FIGS. 3-4. Implant 10 includes a spacer 101, a distal portion 102, and a proximal portion 103. Implant 10 defines a series of openings 105 disposed between distal portion 102 and spacer 101, and proximal portion 103 and spacer 101. Implant 10 includes a series of tabs 106, a pair of which are disposed opposite each other, along the longitudinal axis of implant 10, on either side of each opening 105. Implant 10 also includes wings 107 that may be deployed so they extend radially from implant 10 when it is in the deployed configuration. As illustrated best in FIGS. 3-4, the arrangement of openings 105 and tabs 106 affect the shape and/or size of wings 107. In some embodiments, the opposing tabs 106 can be configured to engage each other when implant 10 is in the deployed configuration, thereby serving as a positive stop to limit the extent that wings 107 are deployed. In other embodiments, for example, the opposing tabs 106 can be configured to engage each other during the deployment process, thereby serving as a positive stop, but remain spaced apart when implant 10 is in the deployed configuration (see, for example, FIGS. 3-4). In such embodiments, the elastic properties of wings 107 can cause a slight “spring back,” thereby causing the opposing tabs 106 to be slightly spaced apart after tabs 106 have been moved to deploy wings 107.

As illustrated best in FIG. 1, when implant 10 is in the collapsed configuration, wings 107 are contoured to extend slightly radially from remaining portions of implant 10. In this manner, wings 107 are biased such that when a compressive force is applied, wings 107 will extend outwardly from spacer 101. Wings 107 can be biased using any suitable mechanism. For example, wings 107 can be biased by including a notch in one or more locations along wing 107. Alternatively, wings 107 can be biased by varying the thickness of wings 107 in an axial direction. In addition, wings 107 can be stressed or bent prior to insertion such that wings 107 are predisposed to extend outwardly when a compressive force is applied to implant 10. In such embodiments, the radius of wings 107 is greater than that of the remaining portions of implant 10 (e.g., the remaining cylindrical portions of implant 10). Preferably, wings 107 adjacent the proximal portion of implant 10 are designed to be predisposed to extend outwardly under less force than wings 107 adjacent the distal portion of implant 10. This arrangement causes the proximal wings to deploy first and thus facilitates the proper location of implant 10 between the desired spinous processes.

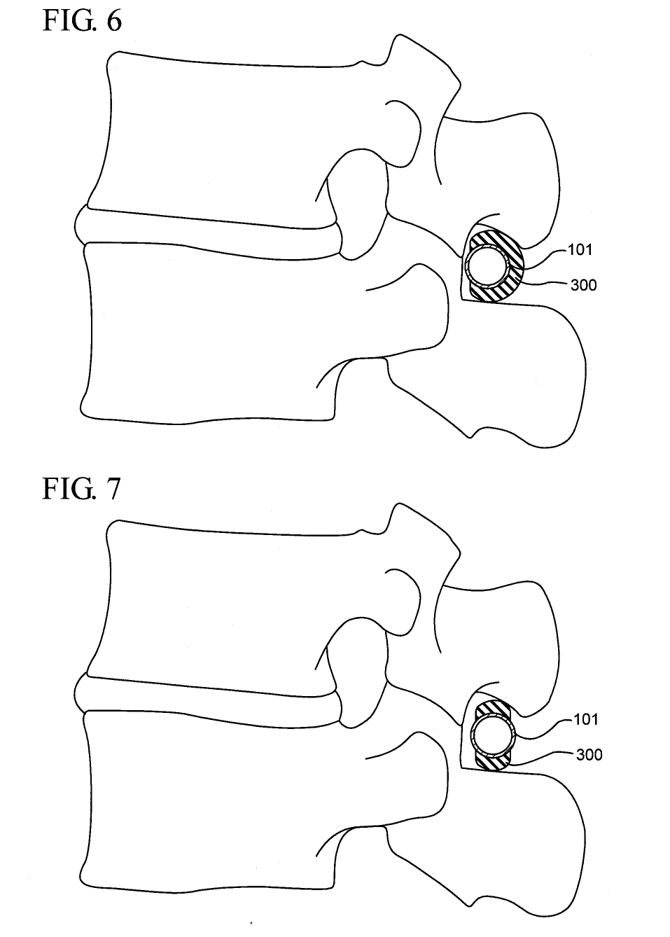

Preferably, implant 10 includes an outer compliant layer 300 located on an outer surface of spacer 101 in the areas where spacer 101 contacts an inferior portion of a superior spinous process and a superior portion of an inferior spinous process. See FIGS. 6 through 9. Alternatively, compliant layer 300 can be located about the entire surface of implant 10 along the entire axial length of implant 10, or along the distal portion 102 and along spacer 101, or along the proximal portion 103 and along spacer 101. Compliant layer 300 may be formed from materials that may have a Modulus of Elasticity (MOE) that is particularly matched with the vertebral members along which implant 10 is located. For example, the difference of the MOE of compliant layer 300 and these vertebral members is not great than about 30 GPa. In other embodiments, the difference is less, such as not greater than about 15 GPa, not greater than about 5 GPa, or not greater than about 1 GPa. Specific examples of the material for compliant layer 300 can include silicone, polyaryletheretherketone (PEEK), polyurethane, and rubber. Other materials may also be used.

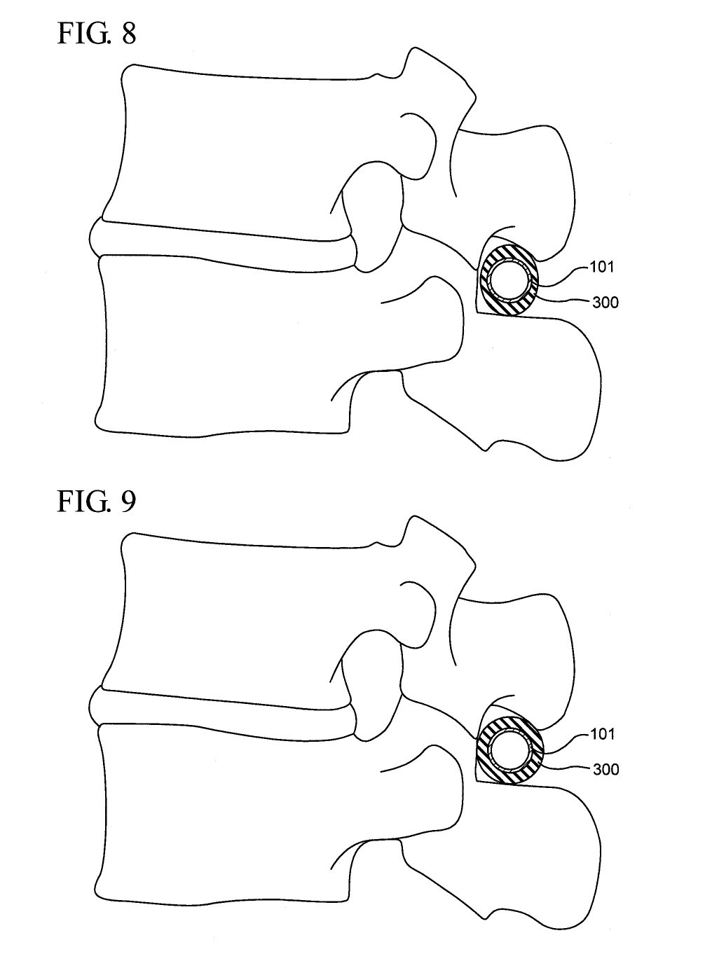

Compliant layer 300 is applied to the outer surface of spacer 101 in such a way that compliant layer 300 has its greatest thickness in the areas where spacer 101 will contact the spinous processes. See FIGS. 6 through 9. In FIG. 6, compliant layer 300 is substantially uniformly disposed around most of the circumference of spacer 101 except along the anterior side of spacer 101. In FIG. 7, compliant layer 300 is disposed along the superior and inferior side of spacer 101. In FIG. 8, compliant layer 300 is disposed around the entire circumference of spacer 101, but the thickness is minimized along the anterior and posterior portions of spacer 101. In FIG. 9, compliant layer 300 is disposed completely and substantially uniformly around the circumference of spacer 101. Preferably, compliant layer is between about 0 and 20 mm thick in these areas. Compliant layer 300 should have a minimal thickness in the area that is disposed along the anterior portion of spacer 101 when spacer 101 is located in the patient between adjacent spinous processes. See, for example, FIG. 8. Alternatively, compliant layer 300 can be non-existent in this area. See FIGS. 6 and 7. In yet another embodiment, compliant layer 300 may be located substantially symmetrically around the circumference of spacer 101. See FIGS. 8 and 9. Where there is no layer 300 along the anterior portion of spacer 101, it can be implanted between adjacent spinous processes as anteriorly as possible. This ensures that spacer 101 (i) is able to provide maximum distraction/spacing between adjacent spinous processes with minimal size, (ii) minimizes the potential for unwanted posterior migration of the implant, and (iii) provides the best potential outcome for the patient. See, for example, FIGS. 6 and 7. Compliant layer 300 can be applied in many different ways. For example, compliant layer 300 may be molded over appropriate portions of implant 10, it may be formed as a separate member and placed over implant 10, or it may be applied by chemically coating implant 10.

Spacer 101 also includes a central body 201 disposed within a lumen 120 defined by spacer 101. Central body 201 is configured to maintain the shape of spacer 101 during insertion, to prevent wings 107 from extending inwardly into a region inside of spacer 101 during deployment and/or to maintain the shape of spacer 101 once it is in its desired position. As such, central body 201 can be constructed to provide increased compressive strength to spacer 101. In other words, central body 201 can provide additional structural support to spacer 101 (e.g., in a direction transverse to the axial direction) by filling at least a portion of the region inside spacer 101 (e.g., lumen 120) and contacting the walls of spacer 101. This can increase the amount of compressive force that can be applied to spacer 101 while allowing it to still maintain its shape and, for example, the desired spacing between adjacent spinous processes. In some embodiments, central body 201 can define a lumen 120, while in other embodiments, central body 201 can have a substantially solid construction. As illustrated, central body 201 is fixedly coupled to spacer 101 with a coupling portion 203, which is configured to be threadedly coupled to the distal portion of spacer 101. The distal end of coupling portion 203 of central body 201 includes an opening 204 configured to receive a tool that is designed to deform the distal end of coupling portion 203. In this manner, once central body 201 is threadedly coupled to spacer 101, coupling portion 203 can be deformed or peened to ensure that central body 201 does not become inadvertently decoupled from spacer 101. In some embodiments, an adhesive, such as a thread-locking compound can be applied to the threaded portion of coupling portion 203 to ensure that central body 201 does not inadvertently become decoupled from spacer 101. Although illustrated as being threadably coupled, central body 201 can be coupled to spacer 101 by any suitable means. In some embodiments, for example, central body 201 can be coupled to spacer 101 by, for example, a friction fit. In other embodiments, central body 201 can be coupled to spacer 101 by an adhesive. Central body 201 can have a length such that central body 201 is disposed within lumen 120 along substantially the entire length of spacer 101 or only a portion of the length of spacer 101 or along a portion of the length of spacer 101 and a portion of proximal portion 103 and/or a portion of distal portion 102.

The proximal portion of central body 201 preferably includes cavity 202 configured to receive a portion of an insertion tool, not shown. Such an insertion tool is similar to the tool shown and described in commonly assign U.S. Patent Application Publication No. 2007/0276493, the entire contents of which are hereby expressly incorporated herein by reference.

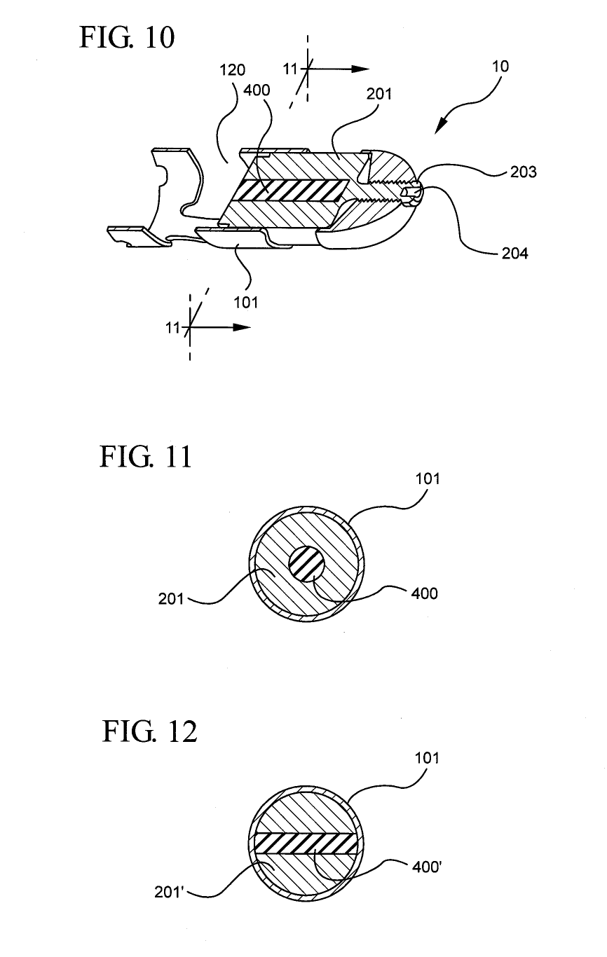

FIG. 10 illustrates an interspinous process device according to another embodiment of the invention. In the embodiment shown in FIG. 10, an inner core 400 is located in cavity 202. Inner core 400 is formed from the same types of material as described above in connection with coating 300. As shown in FIG. 11, inner core 400 may be formed as a cylinder having a generally circular cross section, although the cylinder could have other cross sections as well, such as a polygon or other symmetrical or unsymmetrical geometric shape. In the foregoing examples, inner core 400 is located within cavity 202 such that inner core is completely surrounded by central body 201. Alternatively, the inner core may extend across the diameter of lumen 120 such that central body 201 is disposed along the superior and inferior sides of inner core 400′. See for example, FIG. 12. In this embodiment, inner core 400′ may have a generally rectangular cross section. Alternatively, the inner core could be arranged within lumen 120 so that central body is disposed along the distal and proximal sides of the inner core. As with the embodiment shown in FIG. 11, the cross section of inner core 400′ may take various geometric shapes. Other configurations may be used for the inner core as long as the inner core takes up some of the load on the implant when the spine is in extension.

In use, once implant 10 is positioned on a suitable insertion tool, implant 10 is inserted into the patient's body and disposed therein such that spacer 101 is located between adjacent spinous processes. Thereafter, the insertion tool is used to move central body 201 axially towards the proximal portion of spacer 101 while simultaneously maintaining the position of the proximal portion of spacer 101. In this manner, a compressive force is applied along the longitudinal axis of spacer 101, thereby causing spacer 101 to fold or bend to deploy wings 107 as described above. Similarly, to move spacer 101 from the deployed configuration to the collapsed configuration, the insertion tool is actuated in the opposite direction to impart an axial force on the distal portion of spacer 101 in a distal direction, moving the distal portion distally, and moving spacer 101 to the collapsed configuration.

Although shown and described above without reference to any specific dimensions, in some embodiments, spacer 101 can have a cylindrical shape having a length of approximately 34.5 mm (1.36 inches) and a diameter between 8.1 and 14.0 mm (0.32 and 0.55 inches). In some embodiments, the wall thickness of spacer 101 can be approximately 5.1 mm (0.2 inches).

Similarly, in some embodiments, inner core 201 can have a cylindrical shape having an overall length of approximately 27.2 mm (1.11 inches) and a diameter between 8.1 and 14.0 mm (0.32 and 0.55 inches).

In some embodiments, the shape and size of openings 105 located adjacent the distal portion 102 can be the same as that for the openings 105 located adjacent the proximal portion 103. In other embodiments, the openings 105 can have different sizes and/or shapes. In some embodiments, the openings 105 can have a length of approximately 11.4 mm (0.45 inches) and a width between 4.6 and 10 mm (0.18 and 0.40 inches).

Similarly, the shape and size of tabs 106 can be uniform or different as circumstances dictate. In some embodiments, for example, the longitudinal length of tabs 106 located adjacent proximal portion 103 can be shorter than the longitudinal length of tabs 106 located adjacent distal portion 102. In this manner, as spacer 101 is moved from the collapsed configuration to the deployed configuration, tabs 106 adjacent distal portion 102 will engage each other first, thereby limiting the extent that wings 107 adjacent distal portion 102 are deployed to a greater degree than wings 107 located adjacent proximal portion 103. In other embodiments, the longitudinal length of tabs 106 can be the same. In some embodiments, the longitudinal length of tabs 106 can be between 1.8 and 2.8 mm (0.07 and 0.11 inches). In some embodiments, the end portions of opposing tabs 106 can have mating shapes, such as mating radii of curvature, such that opposing tabs 106 engage each other in a predefined manner.

Although illustrated as having a generally rectangular shape, wings 107 can be of any suitable shape and size. In some embodiments, for example, wings 107 can have a longitudinal length of approximately 11.4 mm (0.45 inches) and a width between 3.6 and 3.8 mm (0.14 and 0.15 inches). In other embodiments, the size and/or shape of wings 107 located adjacent proximal portion 103 can be different than the size and/or shape of tabs 106 located adjacent distal portion 102. Moreover, as described above, wings 107 can be contoured to extend slightly radially from spacer 101. In some embodiments, for example, wings 107 can have a radius of curvature of approximately 12.7 mm (0.5 inches) along an axis normal to the longitudinal axis of spacer 101.

In some embodiments, wings 107 and spacer 101 are monolithically formed. In other embodiments, wings 107 and spacer 101 are formed from separate components having different material properties. For example, wings 107 can be formed from a material having a greater amount of flexibility, while spacer 101 can be formed from a more rigid material. In this manner, wings 107 can be easily moved from the collapsed configuration to the deployed configuration, while spacer 101 is sufficiently strong to resist undesirable deformation when in use.

FIG. 13 shows another interspinous process implant 1000 that may incorporate the spacer 101 of this invention. Implant 1000 includes a first wing 1010, a spacer 101 and a lead-in and distraction guide 1100. Alternatively, implant 1000 may include no lead-in and distraction guide. Implant 1000 may include a second wing 1020 that may be fixed to implant 1000 or may be removably attached thereto. For more a more detailed description, see the disclosure of U.S. Application Publication No. 2008/0039859. As mentioned above, the entire disclosure of that document is hereby expressly incorporated herein by reference. Compliant layer 300 is located around the spacer of FIG. 13 in a similar fashion as described in connection with the previous embodiments of this invention.

FIG. 14 shows yet another interspinous process implant 2000 that may incorporate the compliant layer of this invention. Implant 2000 has a generally H-shaped configuration wherein the cross-bar 2010 of the H is the spacer 101 of this invention. Compliant layer 300 is preferably located along the superior and inferior portions of cross-bar 2010.

Spacer 101 can be constructed with various biocompatible materials such as, for example, titanium, titanium alloy, surgical steel, biocompatible metal alloys, stainless steel, Nitinol, plastic, polyetheretherketone (PEEK), carbon fiber, ultra-high molecular weight (UHMW) polyethylene, biocompatible polymeric materials, etc. The material of spacer 101 can have, for example, a compressive strength similar to or higher than that of bone. In one embodiment, spacer 101, which is placed between the two adjacent spinous processes, is configured with a material having an elastic modulus higher than the elastic modulus of the bone, which forms the spinous processes. In another embodiment, spacer 101 is configured with a material having a higher elastic modulus than the materials used to configure the distal and proximal portions of the implant. For example, spacer 101 may have an elastic modulus higher than bone, while proximal portion 103 and distal portion 102 have a lower elastic modulus than bone. In yet another embodiment, spacer 101 can be configured with material having a higher elastic modulus than inner core 201, e.g. a titanium alloy material or Nitinol, while inner core 201 can be made with a polymeric material. Alternatively, spacer 101 can be configured with a material having a lower elastic modulus than inner core 201, e.g. spacer 101 can be made with a polymeric material while inner core 201 is made with a titanium alloy material.

While various embodiments of the invention have been described above, it should be understood that they have been presented by way of example only, and not limitation. The foregoing description of the various interspinous process implants is not intended to be exhaustive or to limit the invention. Many modifications and variations will be apparent to the practitioner skilled in the art. It is intended that the scope of the invention be defined by the following claims and their equivalents.

What is claimed is:

1. An apparatus, comprising:
a proximal portion;
a distal portion;
a spacer between the proximal portion and the distal portion and configured to be disposed in a space between adjacent spinous processes, the spacer defining a lumen therethrough;
a layer of material disposed along an outer surface of the spacer such that the layer has a first thickness in the areas adjacent to the spinous process and a second thickness in areas remote from the spinous processes; and
a central body configured to be disposed at least partially within the lumen of the spacer, the central body being movable axially relative to the spacer wherein such axial movement moves the spacer between a collapsed configuration and an deployed configuration.

2. The apparatus of claim 1, wherein when in the expanded configuration, the distal portion and the proximal portion each has an outer perimeter greater than an outer perimeter of the spacer.

3. The apparatus of claim 1, wherein the layer is made from a material selected from the group consisting of silicone, polyaryletheretherketone, polyurethane and rubber.

4. The apparatus of claim 3, wherein the first thickness is less than about 20 mm.

5. The apparatus of claim 1, wherein the second thickness is equal to or greater than about 0.

6. The apparatus of claim 1, wherein the first thickness is substantially equal to the second thickness.

7. The apparatus of claim 1, wherein the first thickness is greater than the second thickness.

8. An apparatus, comprising:
a body having a distal portion, a central portion and a proximal portion, wherein the central portion is configured to be disposed in a space between adjacent spinous processes; and
a layer of material disposed along an outer surface of the central portion such that the layer has a first thickness in the areas adjacent to the spinous process and a second thickness in areas remote from the spinous processes.

9. The apparatus of claim 8, wherein the layer is made from a material selected from the group consisting of silicone, polyaryletheretherketone, polyurethane and rubber.

10. The apparatus of claim 8, wherein the first thickness is less than about 20 mm.

11. The apparatus of claim 8, wherein the second thickness is equal to or greater than about 0.

12. The apparatus of claim 8, wherein the first thickness is substantially the same as the second thickness.

13. The apparatus of claim 8, wherein the first thickness is greater than the second thickness.

14. An apparatus, comprising:
a spacer adapted to be disposed in a space between adjacent spinous processes; and
a layer of material disposed along an outer surface of the spacer such that the layer has a first thickness in the areas adjacent to the spinous process and a second thickness in areas remote from the spinous processes.

15. The apparatus of claim 14, wherein the layer is made from a material selected from the group consisting of silicone, polyaryletheretherketone, polyurethane and rubber.

16. The apparatus of claim 15, wherein the first thickness is less than about 20 mm.

17. The apparatus of claim 16, wherein the second thickness is equal to or greater than about 0.

18. The apparatus of claim 14, wherein the first thickness is substantially equal to the second thickness.

19. The apparatus of claim 14, wherein the first thickness is greater than the second thickness.

20. An apparatus, comprising:
an outer shell having a distal portion, a central portion and a proximal portion, wherein the outer shell is configured to be disposed in a space between adjacent spinous processes, the outer shell defining a lumen therethrough; and
a central body configured to be disposed at least partially within the lumen of the outer shell, the central body being movable axially relative to the outer shell wherein such axial movement moves the outer shell between a collapsed configuration and a deployed configuration; and
an inner resilient core.

21. The apparatus of claim 20, wherein the inner resilient core is made from a material selected from the group consisting of silicone, polyaryletheretherketone, polyurethane and rubber.

22. The apparatus of claim 21, wherein the inner resilient core is located adjacent to the central portion.
